(19) United States
(12) Patent Application Publication
(10) Pub. No.: US 2008/0208333 A1
(43) Pub. Date: Aug. 28, 2008

(54) Implantable elastomeric artificial heart with rechargeable battery

(76) Inventor: Ernest John Shearing, Porterville, CA (US)

Correspondence Address: Ernest John Shearing, 297 W. Grand Ave., Porterville, CA, 93257, US

(21) Appl. No.: 11/709,361

(22) Filed: Feb. 22, 2007

(51) Int. Cl. A61M 1/10

(52) U.S. Cl. 623/3.1

(57) ABSTRACT

A new type of artificial heart with rechargeable battery to power solenoid means surrounding the body of the device, said solenoids effecting repulsion or attraction of small permanent magnets on plastic disks which stud the body of the elastomeric artificial heart, and via this means effect mechanical compression or expansion of the chambers of the heart.

### CROSS REFERENCE TO RELATED APPLICATIONS

“Not applicable”.

### STATEMENT REGARDING FEDERALLY SPONSORED RESEARCH OR DEVELOPMENT

“Not Applicable”.

### REFERENCE TO SEQUENCE, ETC.

“Not Applicable”.

### BACKGROUND OF THE INVENTION

Medicine & Health:

Transplantable ARtificial Organs—Differs from the prior art in that electromagnetic coils and magnets are used to effect mechanical contraction of the heart.

### BRIEF SUMMARY OF THE INVENTION

Current through solenoids effects attraction and repulsion of magnets on plastic disks on the body of the artificial heart and thereby effects pumping action which emulates that of the natural heart.

Did not use any other documents or resources other than my own ingenuity and public domain material.

### BRIEF DESCRIPTION OF THE VIEWS OF THE DRAWING

FIG. 1 shows a cross-sectional view of the contemplated embodiment of the invention. The numbers on the drawing refer to the various parts of the elastomeric artificial heart, to wit: 1, is the plastic tube with integral ball and cage valve (not shown) which, in the case of the left atrium, is surgically attached to the pulmonary vein, number 2, refers to the Lycra patch which is sutured to the body of the device. Number three is the left atrium, number 4 shows the wall of the heart and a few of the nylon re-inforcing cords that run through it. Number 5 is the left ventricle, 6 is the outlet for the suture to the aorta with another ball and cage valve inside the outlet (not shown), whereas number 7 is the outlet to the pulmonary artery with a similar set-up and another ball and cage valve (also not shown) for the right atrium. Number 8 is the eye hook and spring which controls the action of the flap valve, #9, and lastly number 10 is the septum of the heart, which may or may not include a plastic insert for additional strength as the actual working in situ may require.

FIG. 2 shows a cross-sectional view of a solenoid, #11, and a permanent magnet, number 12, which is anchored to a flat plastic disk, number 13, which is anchored to the body of the elastomeric artificial heart. There are a number of such units studding the outer surface of the device and it is by means of the reversible current in the solenoids and the attraction and repulsion between the fields of the solenoids and those of the permanent magnets that contraction and expansion of the chambers of the heart is effected, thereby producing a mechanical pumping action that simulates that of the real or natural organ.

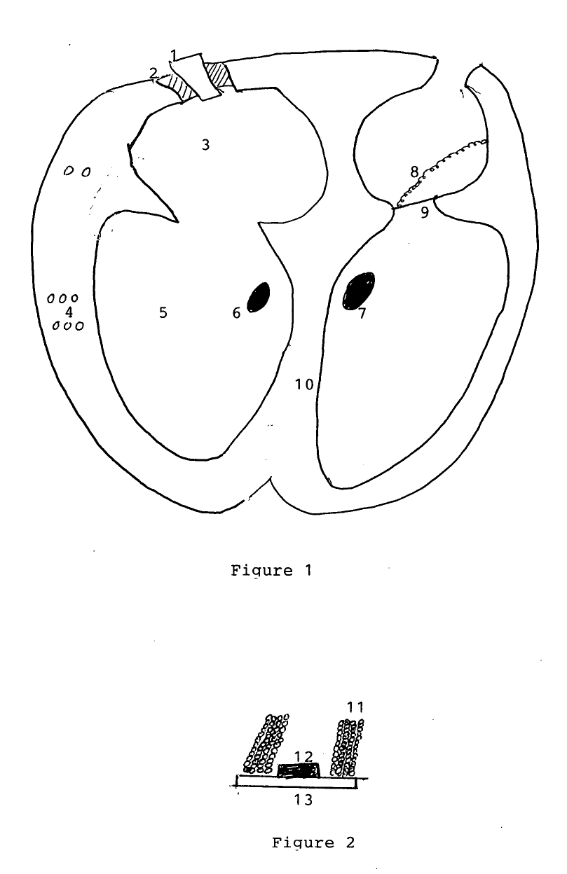

### DETAILED DESCRIPTION OF THE INVENTION

Object of the implant is to save the lives of people with cardiac arrest, bullet wounds, or other irreparable damage to the tissues and/or functioning of the heart, and providing a means of saving the lives of those people who currently can not be saved by current surgical techniques.

The artificial heart is composed of a flexible, elastomeric substance with nylon re-inforcing cords incorporated into the body of the device. If this method proves impractical, then one can use a tightly woven fabric that has been rubberized a la the space suits used by NASA.

The shape of the artificial heart is exactly similar to that of a real heart, and like the actual human heart, it is composed of four chambers, two atria and two ventricles. The right and left atria have two patches of elastic or Lycra sewn onto the two top entryways into each of these respective two chambers. They are attached to two flexible plastic tubes which are sutured to the vena CAVA and the pulmonary veins. Likewise there is a similar set-up for the aorta and the pulmonary artery for the left and right ventricles, respectively.

Ball and cage valves are located inside the two plastic tubes oriented so as to permit inflow of blood but block egress of that fluid during the contractile phase of the heart's cycle

Inside the two atria are nylon screws embedded firmly in the walls of the elastomeric artificial heart. Each nylon screw hooks into a spring which controls a flap valve, which separates the atrium from the ventricle. Upon compression of the atrium, the blood is forced out through the opening between atrium and ventricle as the flap valve opens and the atrium is compressed. The ventricles are filled with blood while the atria empty, upon evacuation, the spring upon the release from its' extension; snaps the flap valve shut, in preparation for the next cycle of filling, compression, expansion, and refilling.

The two ventricles are similarly equipped with outlets for the aorta and the pulmonary artery; these outlets themselves have ball and cage valves oriented so as to prevent backflow of blood when the ventricles are in their contraction phase.

The action of the heart is effected in the following manner. The heart is surrounded on all sides by a number of tightly and multiply wound solenoids, which act like magnetic dipoles and are fitted over small magnets attached to plastic disks on the body of the artificial heart. The heart comes with a fine wire and a microelectrode which is surgically implanted in the vagus nerve so that the body's own natural mechanisms are used to initiate the compression cycle of this device. Upon initiation by an impulse from the vagus nerve, a micro-switch or micro-relay is triggered, which then sends current via a microchip controlled fashion to the solenoids surrounding the appropriate area of the artificial heart. This sets up a magnetic field in the solenoids, which either attracts or repells the underlying magnets attahed to the plastic disks studding the body of the heart. By repulsion, compression and contractile force of the elastomeric artificialheart is effected.

The flexion of the walls of the device drive blood through the body via this electromagnetic-mechanical motion. When the appropriate chamber is drained of blood, a timing circuit in the microchip throws another microswitch, the current is reversed, so that the solenoids now attract the permanent magnets on the Plastic disks and this effects attraction of the said magnets, resulting in an expansion of the walls of the artificial heart, readying it for the next cycle.

To shield the artificial heart from EMI pulses, there is a wire mexh encasing the device, solenoids, and circuitry. Lastly, the wire mesh is itself along with the whole device, encased in a plastic film which prevents corrosion and leaching of the copper wires into the tissues of the body, thus preventing heavy metal poisoning.

This completes the description of the artificial heart as I envision it.

1. “I claim priority for the invention of an artificial heart to utilize electromagnetic means to effect mechanical pumping action of an elastomeric artificial heart with rechargeable battery.”
